(19) United States
(12) Patent Application Publication
(10) Pub. No.: US 2017/0196568 A1
(43) Pub. Date: Jul. 13, 2017

(54) LEFT ATRIAL APPENDAGE CLOSURE

(71) Applicant: RAINBOW MEDICAL LTD., Herzliya (IL)

(72) Inventors: Yossi GROSS, Moshav Mazor (IL); Michael KARDOSH, Kiriat Ono (IL); Amir KISHON, Petach Tikva (IL); Ido SADAN, Tel Aviv (IL); Jonathan YALOM, Tel Aviv (IL); Zev SOHN, Ginot Shomron (IL); Omri GINO, Herzliya (IL); Eliachar ELIAHU, Haifa (IL)

(73) Assignee: RAINBOW MEDICAL LTD., Herzliya (IL)

(21) Appl. No.: 15/324,429

(22) PCT Filed: Jul. 7, 2015
(86) PCT No.: PCT/IB2015/055132

§ 371 Date: Jan. 6, 2017

Related U.S. Application Data
(60) Provisional application No. 62/021,327, filed on Jul. 7, 2014.

(51) Int. Cl. A61B 17/128; A61B 17/122; A61B 17/04

(52) U.S. Cl. CPC A61B 17/1285; A61B 17/0469; A61B 17/122; A61B 2017/0496

(57) ABSTRACT

Apparatus (610) for closure of a left atrial appendage (220) of a heart, comprising a deployment device (520) configured to enter a pericardial region of the heart and to be advanced toward an outer surface of the left atrial appendage; and first and second rigid longitudinal rod elements (630) coupled to each other by at least one coupling suture (552), the first and second rigid longitudinal rod elements (630) being deployable by the deployment device on opposing external surfaces of the left atrial appendage, and configured to compress tissue of the left atrial appendage due to pulling of at least one proximal portion of the coupling suture (552). Other applications are also described.

### CROSS-REFERENCE TO RELATED APPLICATIONS

The present application:

(a) claims the priority of U.S. Provisional Application No. 62/021,327, entitled “Left atrial appendage closure,” filed Jul. 7, 2014,

(b) is related to U.S. Provisional Application No. 61/988,457, entitled “Pericardial Access Device,” filed May 5, 2014, and

(c) is related to U.S. application Ser. No. 14/324,457, entitled “Pericardial Access Device,” filed Jul. 7, 2014, and

(d) is related to U.S. application Ser. No. 14/704,857, entitled “Pericardial Access Device,” filed May 5, 2015, and

(d) is related to PCT/IB2015/053280, entitled “Pericardial Access Device,” filed May 5, 2015.

Each of the above applications is incorporated herein by reference.

### FIELD OF THE INVENTION

Applications of the present invention relate generally to cardiac procedures and specifically to apparatus and methods for closing a left atrial appendage in a minimally-invasive manner.

### BACKGROUND

Atrial fibrillation is a common cardiac arrhythmia involving the atria of the heart. During atrial fibrillation, the atria beat irregularly and out of coordination with the ventricles of the heart. Atrial fibrillation disrupts efficient beating of the heart and may result in blood clotting in the atrium leading to an increased risk of a stroke.

Blood clot formation in patients suffering from atrial fibrillation frequently originates in the left atrial appendage. Stagnant blood in the left atrial appendage may form blood clots that can travel through the arterial system, restricting blood flow and causing a stroke.

Left atrial appendage occlusion, or closure, is a treatment strategy for preventing blood flow (and blood clots) from entering or leaving the left atrial appendage. Left atrial appendage occlusion, or closure, is particularly suited for atrial fibrillation patients who are not treated by oral anti-coagulation therapy for various reasons (such as intolerance and/or an increased risk of bleeding).

Several techniques exist for performing left atrial appendage closure.

For example, U.S. Pat. No. 8,721,663 to Kaplan et al. describes methods and apparatus for closing a left atrial appendage. The methods rely on introducing a closure tool from a location beneath the rib cage, over an epicardial surface, and to the exterior of the left atrial appendage. The closure device is described as then being used to close the left atrial appendage, preferably at its base, by any one of a variety of techniques. A specific technique using graspers and a closing loop is illustrated.

An additional example is U.S. Pat. No. 8,663,245 to Francischelli, which describes a system for occluding a left atrial appendage of a patient. The system is described as including a ring occluder that can be positioned around the left atrial appendage and a ring applicator to position the ring occluder with respect to the left atrial appendage. The system is also described as providing a tissue-grasping tool that is separable from the ring applicator tool.

### SUMMARY OF THE INVENTION

Applications of the present invention provide closure apparatus and methods for closing a left atrial appendage. Closing of the left atrial appendage (LAA) in order to separate the inner cavity of the LAA from the interior of the left atrium typically inhibits blood clots that may form in the LAA from entering the left atrium and the arterial system.

In accordance with some applications of the present invention, apparatus for closing the left atrial appendage (LAA) is typically advanced percutaneously, typically through a subxiphoid incision, toward a heart of the subject.

The apparatus for closing the left atrial appendage (LAA) is typically introduced into the pericardial region by a delivery tool configured for accessing the pericardial region. Typically, but not necessarily, the delivery tool is passed over a guidewire that was advanced into the pericardial region by apparatus configured for accessing the pericardial region. Some apparatus and methods for accessing the pericardial region, and for use with the apparatus for closing the left atrial appendage (LAA), are described hereinbelow.

Typically, apparatus for accessing the pericardial region (such as is described hereinbelow in the Detailed Description) is advanced towards the heart under the guidance of an intracorporeal imaging device and comprises means for applying suction to the pericardium and puncturing thereof in order to penetrate the pericardium and access the pericardial region. Once the pericardium is penetrated to access the pericardial region, a guidewire is placed in the pericardial region and a delivery tool can then be passed over the guidewire for delivery of closure apparatus for facilitating left atrial appendage (LAA) closure.

For some applications, apparatus for accessing the pericardial region comprises a sheath which is advanced to the pericardium, and the left atrial appendage (LAA) closure apparatus is delivered into the pericardial region through the sheath.

For other applications, the apparatus for accessing the pericardial region does not comprise a sheath, but rather is shaped to define a suction port configured to facilitate drawing a portion of the pericardium through the suction port. The apparatus for accessing the pericardial region comprises means for puncturing the portion of the pericardium that is drawn into the suction port in order to gain access to the pericardial region. Once the pericardium is penetrated to access the pericardial region, a guidewire is placed in the pericardial region and a delivery tool can then be passed over the guidewire for delivery of closure apparatus for facilitating left atrial appendage (LAA) closure.

There is therefore provided, in accordance with some applications of the present invention, apparatus for closure of a left atrial appendage of a heart including:

a deployment device; and

first and second longitudinal rod elements coupled to each other by at least one coupling suture, the first and second longitudinal rod elements being deployable by the deployment device on opposing external surfaces of the left atrial appendage, and configured to compress tissue of the left atrial appendage due to pulling of at least one proximal portion of the coupling suture.

For some applications, the apparatus further includes a delivery tool configured in shape and size to enter a pericardial region of the heart and to be advanced toward an outer surface of the left atrial appendage, the deployment device is configured to be advanced into the pericardial region through the delivery tool.

For some applications, the deployment device includes two deployment rods configured to deploy the first and second longitudinal rod elements on opposing external surfaces of the left atrial appendage.

For some applications, the apparatus further includes a stabilizing tool advanceable into a pericardial region, and shaped and arranged to stabilize tissue of the left atrial appendage.

For some applications, the stabilizing tool is configured to stabilize tissue of the left atrial appendage by applying suction to the tissue.

For some applications, the stabilizing tool includes a mechanical grasping element that is configured to stabilize tissue of the left atrial appendage by grasping the tissue.

For some applications, the stabilizing tool is configured to move the tissue of the left atrial appendage to position the tissue through the first and second longitudinal rod elements.

For some applications, the first and second rod elements are each shaped to define flat tissue-contact surfaces.

For some applications, the first and second rod elements are each shaped to define rounded tissue-contact surfaces.

For some applications, the first and second rod elements include rigid first and second rod elements.

There is therefore provided, in accordance with some applications of the present invention, a method for closing a left atrial appendage of a heart of a subject, including:

percutaneously advancing a deployment device into a pericardial region of the heart toward an outer surface of the left atrial appendage;

using a deployment device, deploying first and second rigid longitudinal rod elements on opposing outer surfaces of the left atrial appendage, the first and second rod elements coupled to each other by at least one coupling suture; and

closing the left atrial appendage by pressing the first and second rod elements against a base portion of the left atrial appendage, by pulling a proximal portion of the coupling suture.

For some applications, the method further includes stabilizing tissue of the left atrial appendage with a stabilizing tool.

For some applications, stabilizing tissue of the left atrial appendage with the stabilizing tool includes stabilizing the tissue by applying suction to the tissue through the stabilizing tool.

For some applications, stabilizing tissue of the left atrial appendage with the stabilizing tool includes stabilizing the tissue by grasping the tissue with the stabilizing tool.

For some applications, the deployment device includes at least one deployment rod and using the deployment device includes using the at least one deployment rod.

For some applications, advancing the deployment device into the pericardial region includes advancing the deployment device under the guidance of an intracorporeal imaging device.

For some applications, deploying the first and second rigid longitudinal rod elements on opposing outer surfaces of the left atrial appendage includes deploying the rod elements in a disposition that is not parallel to an axis of protrusion of the left atrial appendage.

There is therefore provided, in accordance with some applications of the present invention, apparatus for closure of a left atrial appendage of a heart, including:

a stabilizing tool advanceable into a pericardial region, and shaped and arranged to stabilize tissue of the left atrial appendage;

a closure-suture tube configured to enter the pericardial region and to be advanced toward an outer surface of the left atrial appendage;

a closure suture advanceable out of the closure-suture tube and shaped to define a closure loop configured to close the left atrial appendage by tightening around a base portion of the left atrial appendage;

a control tube configured to enter the pericardial region and to be advanced toward an outer surface of the left atrial appendage; and

at least one control suture advanceable out of the control tube and shaped to define a control loop which is disposed in a disposition looped through the closure suture to facilitate placement of the closure suture at the base portion of the left atrial appendage by the control loop pulling the closure loop.

For some applications, the stabilizing tool is configured to stabilize tissue of the left atrial appendage by applying suction to the tissue.

For some applications, the stabilizing tool includes a mechanical grasping element and is configured to stabilize tissue of the left atrial appendage by grasping the tissue.

For some applications, the apparatus further includes a delivery tool configured in shape and size to enter a pericardial region of the heart and to be advanced toward an outer surface of the left atrial appendage, the apparatus is configured to be advanced into the pericardial region through the delivery tool.

There is therefore provided, in accordance with some applications of the present invention, a method for closing a left atrial appendage of a heart, including:

percutaneously advancing a stabilizing tool into a pericardial region of the heart;

stabilizing tissue of the left atrial appendage with the stabilizing tool;

deploying a closure suture loop around the left atrial appendage, the closure suture loop having a control suture loop looped therethrough;

positioning the closure suture loop around a base portion of the left atrial appendage by pulling the closure suture loop using the at least one control suture loop; and

closing the left atrial appendage by tightening the closure suture around the base portion of the left atrial appendage.

For some applications, advancing the stabilizing tool into the pericardial region includes advancing the stabilizing tool under the guidance of an intracorporeal imaging device.

For some applications, stabilizing tissue of the left atrial appendage with the stabilizing tool includes stabilizing the tissue by applying suction to the tissue through the stabilizing tool.

For some applications, stabilizing tissue of the left atrial appendage with the stabilizing tool includes stabilizing the tissue by grasping the tissue with the stabilizing tool.

There is therefore provided, in accordance with some applications of the present invention, apparatus for closure of a left atrial appendage of a heart, including:

a longitudinal deployment device movable through tissue of the left atrial appendage; and

an expandable frame coupled to the deployment device and including first and second compression surfaces, the first and second compression surfaces being deployable by the deployment device on opposing external surfaces of the left atrial appendage, and configured to compress the tissue of the left atrial appendage, subsequently to the deployment device being moved through the tissue.

For some applications, the deployment device is configured to pierce tissue of a left atrial appendage.

For some applications, the first and second compression surfaces each include two or more spokes.

There is therefore provided, in accordance with some applications of the present invention, apparatus for closure of a left atrial appendage of a heart, including:

a deployment device; and

a clip element shaped to define two longitudinal arms coupled to each other by at least one suture, the two longitudinal arms being deployable by the deployment device on opposing external surfaces of the left atrial appendage, and configured to close over tissue of the left atrial appendage due to pulling of the suture.

For some applications, the apparatus further includes:

a delivery tool in which the deployment device is disposed; and

a stabilizing tool disposed within the deployment device, advanceable into a pericardial region of the heart, and shaped and arranged to stabilize the tissue of the left atrial appendage during deployment of the two longitudinal arms on the opposing external surfaces of the left atrial appendage.

For some applications, the stabilizing tool is configured to stabilize tissue of the left atrial appendage by applying suction to the tissue.

For some applications, the stabilizing tool includes a mechanical grasping element and is configured to stabilize tissue of the left atrial appendage by grasping the tissue.

There is therefore provided, in accordance with some applications of the present invention, a method including:

under the guidance of an intracorporeal imaging device, percutaneously advancing a stabilizing tool into a pericardial region of the subject;

stabilizing tissue of the left atrial appendage with the stabilizing tool;

deploying a clip element, the clip element including (a) two rigid longitudinal elements and (b) a cable-tie element having a ratchet, around a portion of the left atrial appendage by aligning the clip element not parallel to an axis of protrusion of the left atrial appendage; and

closing the left atrial appendage by tightening the clip element around the portion of the left atrial appendage by tightening the ratchet by moving at least one of the rigid longitudinal elements toward the other rigid longitudinal element.

There is therefore provided in accordance with some applications of the present invention, apparatus for closure of a left atrial appendage of a heart, including:

a deployment device; and

first and second longitudinal rod elements coupled to each other by at least one coupling suture, the first and second longitudinal rod elements being deployable by the deployment device on external surfaces of the left atrial appendage, and configured to compress tissue of the left atrial appendage due to pulling of at least one proximal portion of the coupling suture.

For some applications, the apparatus further includes a delivery tool configured in shape and size to enter a pericardial region of the heart and to be advanced toward an outer surface of the left atrial appendage, the deployment device is configured to be advanced into the pericardial region through the delivery tool.

For some applications, the deployment device includes two deployment rods configured to deploy the first and second longitudinal rod elements on external surfaces of the left atrial appendage.

For some applications, the apparatus further includes a stabilizing tool advanceable into a pericardial region, and shaped and arranged to stabilize tissue of the left atrial appendage.

For some applications, the stabilizing tool is configured to stabilize tissue of the left atrial appendage by applying suction to the tissue.

For some applications, the stabilizing tool includes a mechanical grasping element that is configured to stabilize tissue of the left atrial appendage by grasping the tissue.

For some applications, the stabilizing tool is configured to move the tissue of the left atrial appendage to position the tissue through the first and second longitudinal rod elements.

For some applications, the first and second rod elements are each shaped to define flat tissue-contact surfaces.

For some applications, the first and second rod elements are each shaped to define rounded tissue-contact surfaces.

For some applications, the first and second rod elements include rigid first and second rod elements.

The present invention will be more fully understood from the following detailed description of embodiments thereof, taken together with the drawings, in which:

### BRIEF DESCRIPTION OF THE DRAWINGS

FIG. 1A-E are schematic illustrations of apparatus for accessing a pericardial region for use with a left atrial appendage (LAA) closure apparatus, in accordance with some applications of the present invention;

FIG. 2 is a schematic illustration of apparatus that creates a working space between two layers of tissue, for use with a left atrial appendage (LAA) closure apparatus, in accordance with some applications of the present invention;

FIGS. 3A-D are schematic illustrations of LAA closure apparatus provided in accordance with some applications of the present invention;

FIGS. 4A-E are schematic illustrations of additional LAA closure apparatus, as provided in accordance with some applications of the present invention;

FIGS. 5A-D are schematic illustrations of several views of additional LAA closure apparatus, as provided in accordance with some applications of the present invention;

FIGS. 6A-C are schematic illustrations of additional LAA closure apparatus, as provided in accordance with some applications of the present invention; and

FIG. 7A-B are schematic illustrations of two respective clip elements, configured for closing a left atrial appendage (LAA), in accordance with some applications of the present invention.

### DETAILED DESCRIPTION OF APPLICATIONS

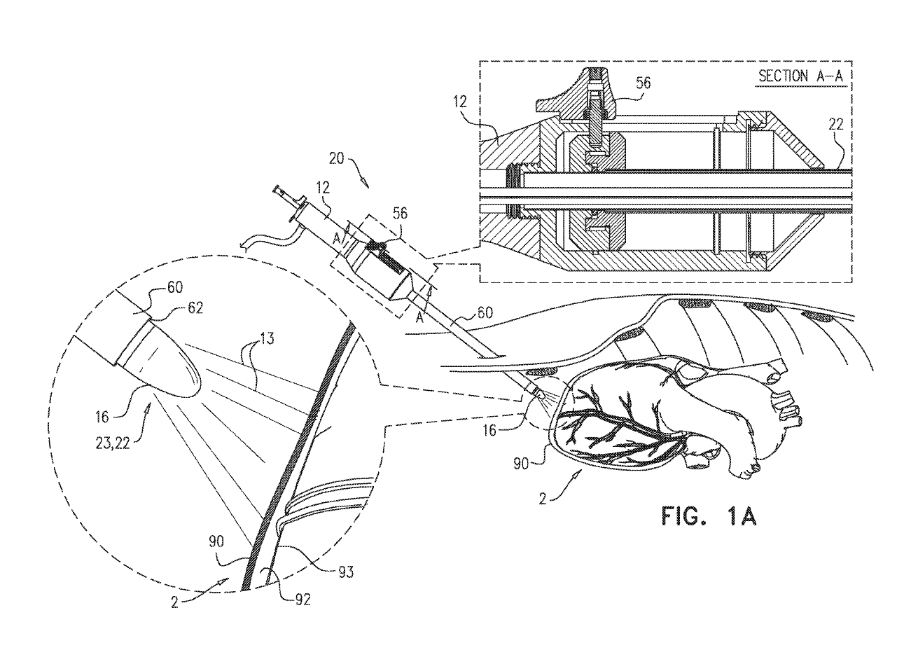

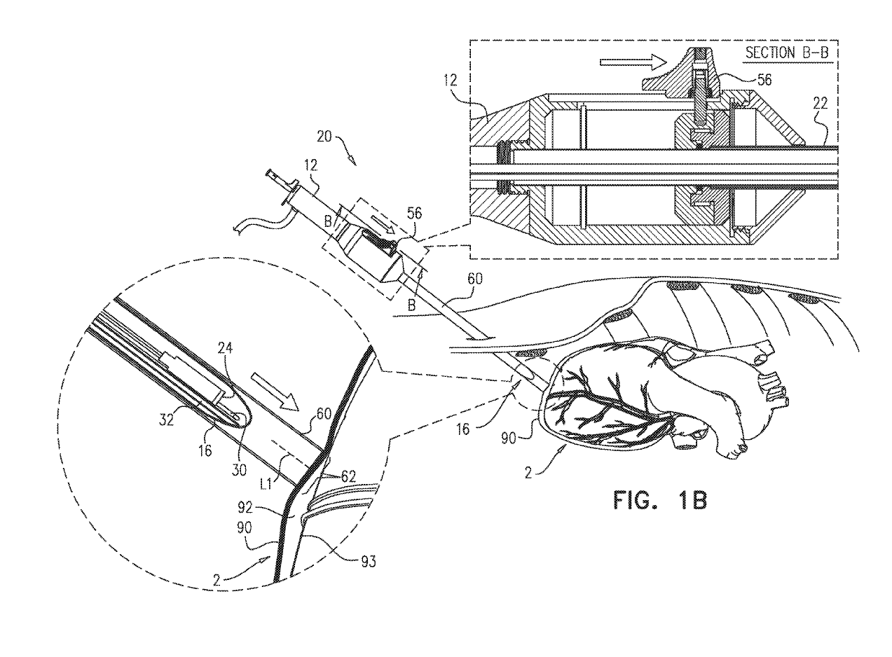

In accordance with some applications of the present invention, apparatus and methods for closing the left atrial appendage (LAA) are provided. The LAA closure apparatus is typically advanced into the pericardial region, following accessing of the pericardial region by use of apparatus for accessing the pericardium. The present description begins with a general overview of apparatus and method for accessing the pericardial region, in accordance with some applications of the present invention, as depicted in FIGS. 1A-E.

FIGS. 1A-E show use of apparatus 20 for penetrating a pericardium 90 of a subject, and accessing a pericardial region 92. FIG. 1A shows a longitudinal guide member 23, e.g., a guide tube 22, of apparatus 20 being distally advanced by blunt dissection toward a heart 2 of the subject, a distal end 16 of guide tube 22 emerging from a distal suction port 62 of sheath 60. Advancement of guide tube 22 may be facilitated by an imaging device 24 (FIG. 1B). Typically, at least one illumination-providing element (not shown) provides illumination for imaging device 24, the illumination being depicted in FIG. 1A by light rays 13.

When guide tube 22 reaches the heart, the operating physician deploys sheath 60 over the guide tube, e.g., by sliding a slide bar 56 distally, and a perimeter of suction port 62 contacts an outer surface of pericardium 90 (FIG. 1B). Suction is then applied to the pericardium through suction port 62, e.g., via a suction tube 30, and a portion of pericardium 90 is drawn into the sheath, as shown in FIG. 1C. (Suction may be applied using a hospital suction generator, and/or an external vacuum pump, and/or a syringe.) For some applications, pericardial tissue is drawn at least 4 mm into sheath 60, e.g., at least 1 cm or at least 1.5 cm into sheath 60.

After the portion of pericardium 90 is drawn into sheath 60, a puncturing element 50 (e.g., a needle 51) is advanced distally to puncture the portion of the pericardium, as shown in FIG. 1D. The puncturing of the portion of the pericardium provides access to pericardial region 92, e.g., a region between pericardium 90 and myocardial tissue 93. Optionally, grasping elements, such as forceps and/or other types of grasping elements (e.g., corkscrew-like or screw-shaped grasping elements), may be employed to grip the portion of pericardium that is inside the sheath, to facilitate the puncturing.

For some applications, following the puncturing of pericardium 90, a guidewire 70 is advanced through a lumen of needle 51 and into pericardial region 92 (FIG. 1E). Typically, the needle is then withdrawn, and a delivery tool carrying the apparatus for closing the left atrial appendage (LAA) is passed over the guidewire and into the pericardial region. Alternatively or additionally, a tube (not shown) is passed over the guidewire, the guidewire is withdrawn, and the delivery tool is passed through the tube and into the pericardial region.

It is noted that the scope of the present invention includes accessing pericardial region 92 by use of other configurations or functional equivalents of apparatus 20. For example, for some applications, apparatus 20 does not comprise a sheath, but rather the longitudinal guide member is shaped to define a suction port at a distal portion of the longitudinal guide member. For such applications, the apparatus facilitates drawing the portion of a pericardium of the heart through the suction port and into the longitudinal guide member (application not shown).

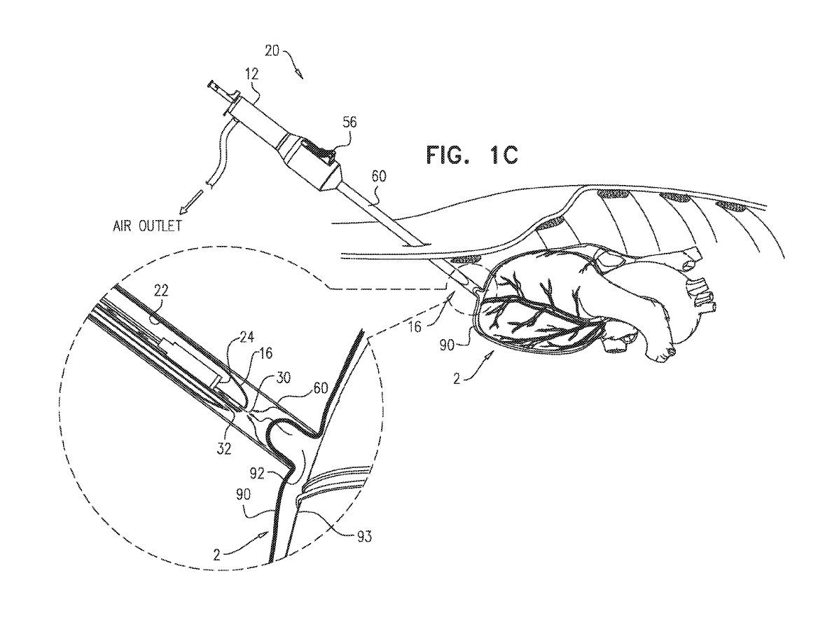

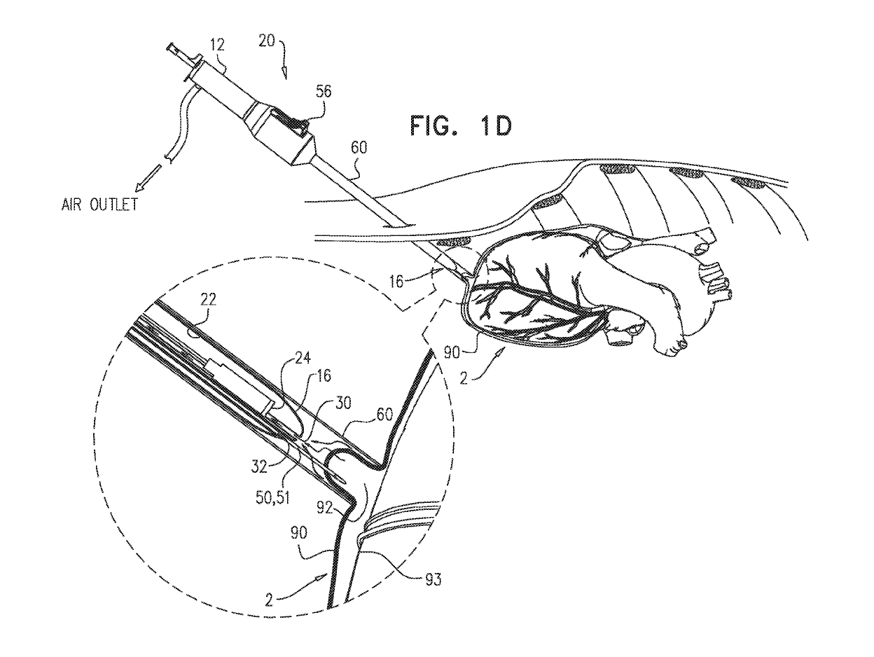

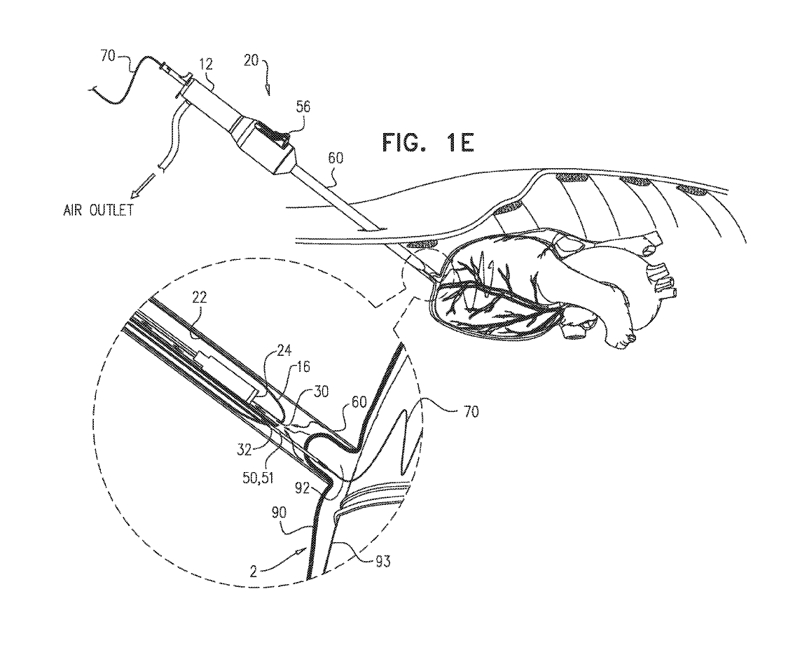

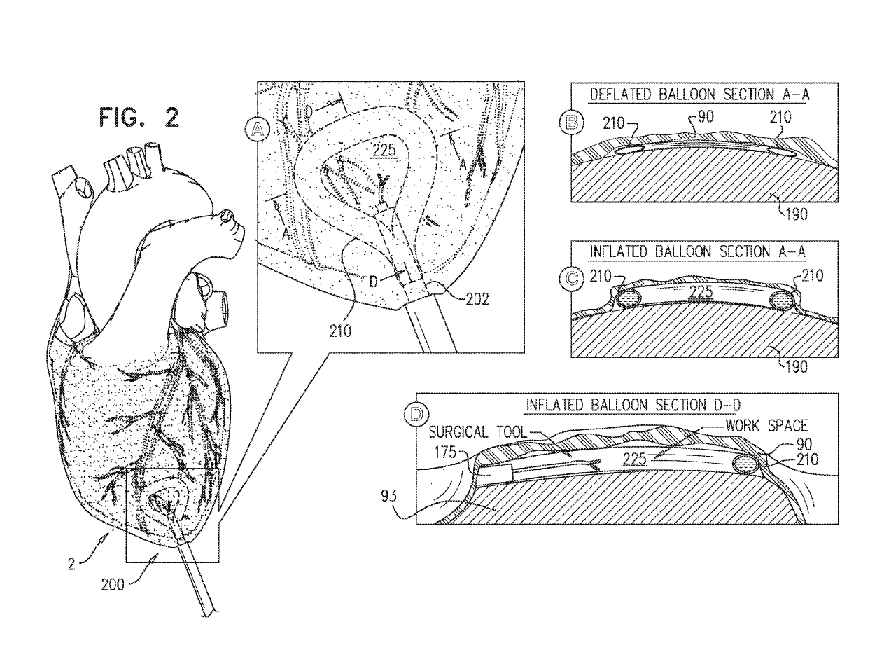

Reference is now made to FIG. 2, which is a schematic illustration of apparatus 200 that creates a working space between two layers of tissue, e.g., between the pericardium and myocardium (i.e., within pericardial region 92), in accordance with some applications of the present invention. In applications in which a working space is created, a surgical tool may then be passed over guidewire 70 (FIG. 1E) and into the working space, in order to perform a surgical procedure on the heart. Creating a working space within a pericardial region, as described in the present application, is useful for facilitating various cardiac procedures, including left atrial appendage (LAA) treatment.

Apparatus 200 for providing a working space within the pericardial region may be used to facilitate left atrial appendage (LAA) closure procedures described herein. Apparatus 200 may be used additionally or alternatively to use of the apparatus for penetrating the pericardium in order to access the pericardial region as described hereinabove with reference to FIGS. 1A-E. Apparatus 200 is typically used once access to the pericardial region has been achieved by apparatus 20 or by any other means. As described hereinabove, apparatus 200 may be advanced over guidewire 70.

Reference is still made to FIG. 2, which shows apparatus 200 that creates a working space 225 between two layers of tissue, e.g., between the pericardium and myocardium (i.e., within pericardial region 92). Apparatus 200 typically comprises a flexible longitudinal element 202 shaped to define a lumen thereof, and an expandable element 210 (e.g., an expandable mesh, and/or an inflatable element) disposed at a distal portion of flexible longitudinal element 202. Expandable element 210 is shaped to define and at least partly surround working space 225, upon the expandable element being expanded. For some applications, apparatus 200 further comprises a surgical tool 175 shaped to be passable through the lumen of the flexible longitudinal element and into the working space.

FIG. 2 depicts a method for performing a procedure in an area between two layers of tissue, such as in pericardial region 92 (FIG. 1E), which is between the pericardium and myocardium. Working space 225 is created by expanding expandable element 210 in the area such that the expandable element defines and at least partly surrounds the working space. Tool 175 is passed into the working space, and is used to perform the procedure. In accordance with some applications of the present invention, tool 175 that is passed into pericardial region 92 includes left atrial appendage (LAA) closure apparatus as described hereinbelow.

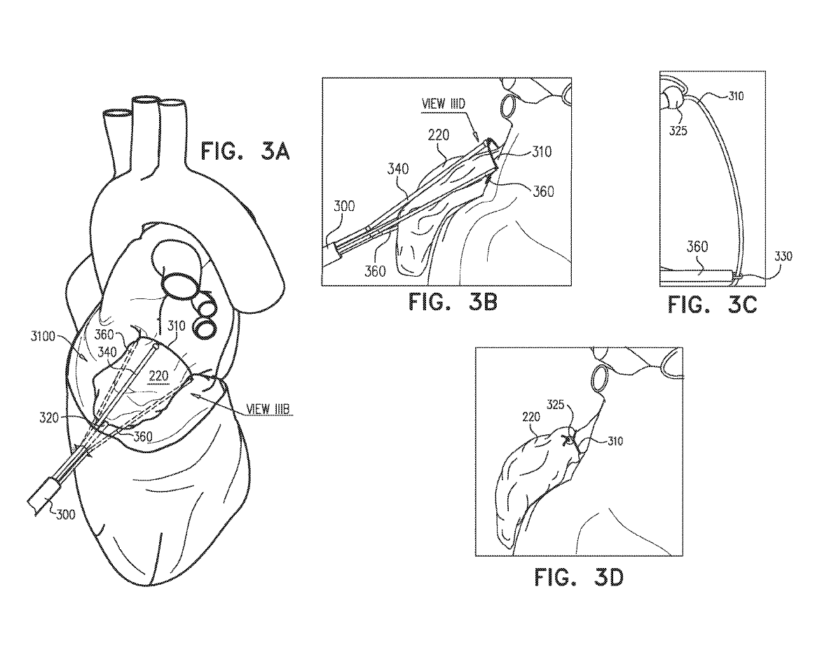

The following description relating to FIGS. 3A-7B describes several examples of closure apparatus, for delivery into the pericardial region of a subject for closing the left atrial appendage (LAA), in accordance with some applications of the present invention. The various closure apparatus described with reference to FIGS. 3A-7B, are advanced into the pericardial region, once the pericardial region has been accessed by use of apparatus for penetrating the pericardium, as described herein with reference to FIGS. 1A-E. Additionally or alternatively, apparatus 200 shown in FIG. 2 for providing a working space within a pericardial region may be used to facilitate left atrial appendage (LAA) closure procedures described herein.

Reference is now made to FIGS. 3A-D, which are schematic illustrations of different perspectives of closure apparatus 3100, provided in accordance with some applications of the present invention. Closure apparatus 3100 is typically delivered by a delivery tool 300. Delivery tool 300 is typically advanced into the pericardial region following accessing the pericardial region with apparatus for accessing the pericardium region, as described hereinabove with reference to FIG. 1A-E. Typically, delivery tool 300 is advanced over a guidewire that was placed in the pericardial region by techniques shown in FIGS. 1A-E. For some applications, delivery tool 300 is passed into the pericardial region by apparatus 200 described in FIG. 2.

In addition to closure apparatus 3100, a stabilizing tool 320 is advanced by delivery tool 300 into the pericardial region of the subject. Stabilizing tool 320 is shaped and arranged to stabilize tissue of left atrial appendage 220 during deployment of the closure apparatus, typically by grasping the tissue or applying suction thereto.

For some applications, stabilizing tool 320 comprises a mechanical grasping element for grasping tissue of the LAA, for example, forceps or graspers. Alternatively, the stabilizing tool comprises a suction-applying element which holds tissue of LAA 220 by applying suction thereto.

A closure suture tube 340 is advanced by delivery tool 300 into the pericardial region. Closure suture tube 340 enters the pericardial region and is advanced toward an outer surface of left atrial appendage 220. A closure suture 310 is advanced out of (or otherwise coupled to) the distal end of closure-suture tube 340 and is shaped to define a closure loop configured to close left atrial appendage 220 by tightening around the base portion of the left atrial appendage.

At least one control tube 360 (e.g., two, as shown in FIGS. 3A-D) enters the pericardial region and is advanced toward an outer surface of left atrial appendage 220. Typically, control tube 360 is advanced by delivery tool 300 into the pericardial region.

At least one control suture is coupled to the distal end of control tube 360 and shaped to define a control loop 330 (shown in an exploded view in FIG. 3C). The control suture is disposed in a disposition looped through the closure suture loop 310 to facilitate placement of the closure suture loop 310 at the base portion of the left atrial appendage by the control loop 330 pulling the closure loop 310 and/or by movement of control tube 360 toward the base portion of the LAA while control loop 330 is holding closure suture loop 310 near the distal end of control tube 360.

Pulling by control loop 330 of closure suture loop 310 typically facilitates placement of closure suture 310 at the base portion of LAA 220. Since control loop 330 is looped through closure suture loop 310, pulling by control loop 330 causes movement of closure suture 310 such that closure suture 310 is moved towards the base portion of LAA 220.

Once closure suture loop 310 is properly disposed around the base portion of the LAA, loop 310 is tightened (and the LAA is thereby closed) by pulling on the ends of loop 310 that are accessible from the proximal end (not shown) of closure-suture tube 340. A knot or bead 325 (or other fixing mechanism) is typically passed through closure-suture tube 340 to maintain the tightness of suture loop 310 around the LAA. It is noted that suture 310 typically does not cut into tissue of LAA 220 and does not damage the tissue.

Once closure suture loop 310 is securely placed around LAA 220, each control loop 330 is disengaged from closure suture 310 by remotely pulling at a single end of the control suture. Stabilizing tool 320 and tubes 340 and 360 are also retracted. Closure suture loop 310 remains properly disposed around the base portion of the LAA, and bead 325 typically maintains the tightness of suture loop 310 around the LAA (FIG. 3D).

For some applications, closure suture loop 310 is shaped to define a flat tissue-contact surface, in order to achieve good contact with tissue of the LAA while pressing against it with the suture. Additionally, the flat tissue-contact surface typically inhibits any risk of cutting into tissue of the LAA by the suture, and thereby reduces the risk of injury to tissue of the LAA. (For other applications, e.g., if closure suture loop 310 is not tightly tied around the LAA, the closure suture is round in cross section.)

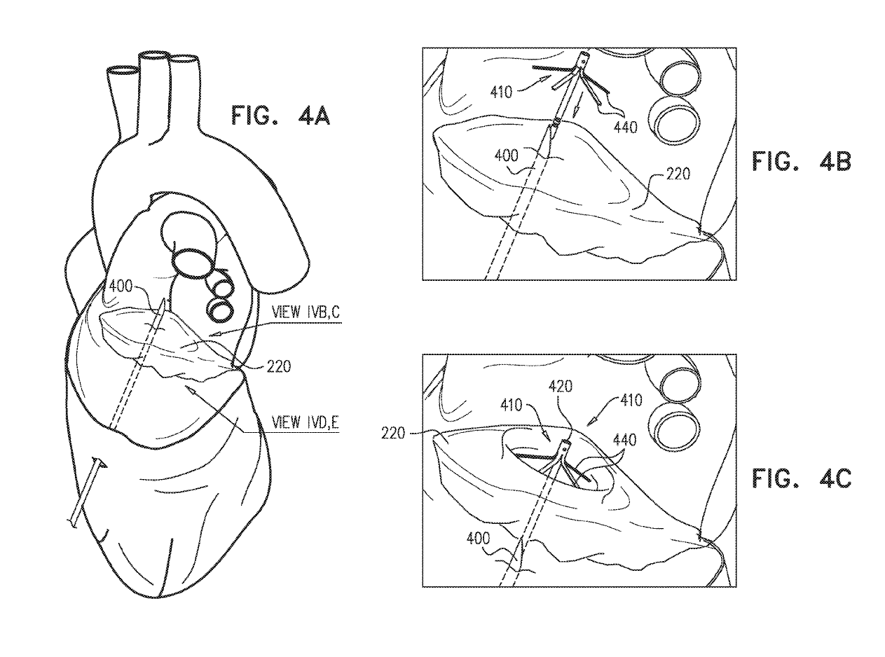

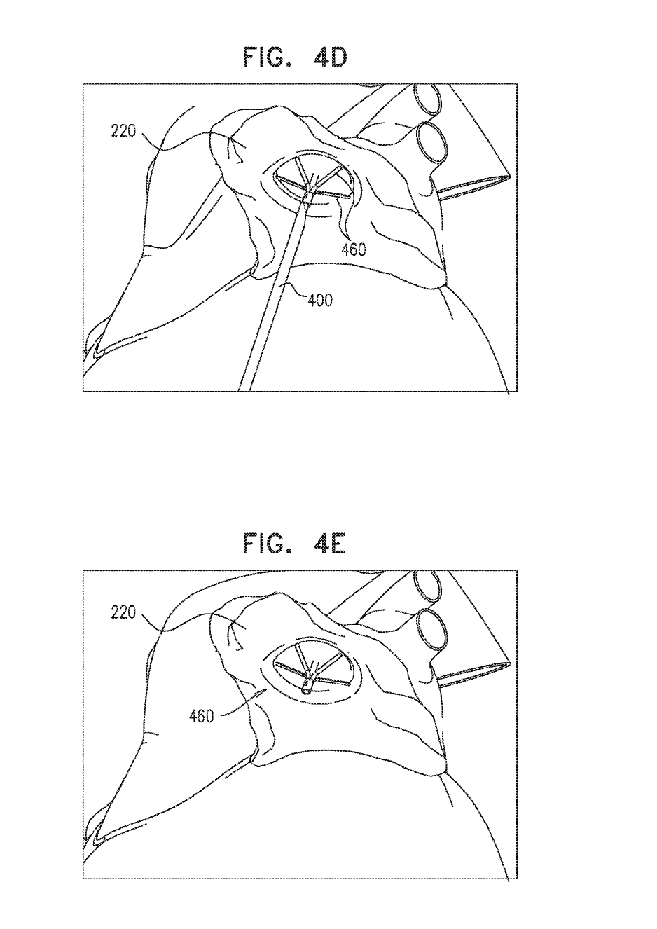

Reference is now made to FIGS. 4A-E, which are schematic illustrations of additional LAA closure apparatus 410, as provided in accordance with some applications of the present invention.

Apparatus 410 comprises a longitudinal deployment device 400 which is moved through tissue of the left atrial appendage. Typically, longitudinal deployment device 400 pierces tissue of LAA 220.

An expandable frame 420 is coupled to deployment device 400 and comprises first and second compression surfaces 440 and 460. Compression surfaces 440 and 460 are deployed by deployment device 400 on opposing external surfaces of left atrial appendage 220, as shown in FIGS. 4A-E. Compression surfaces 440 and 460 compress and squeeze tissue of LAA 220, thereby closing the LAA and excluding the LAA from the left atrium.

FIGS. 4A-E show first and second compression surfaces 440 and 460 as spokes or ribs by way of illustration and not limitation. It is noted that compression surfaces 440 and 460 may be shaped to define full compressing plates that apply a squeezing force to the LAA.

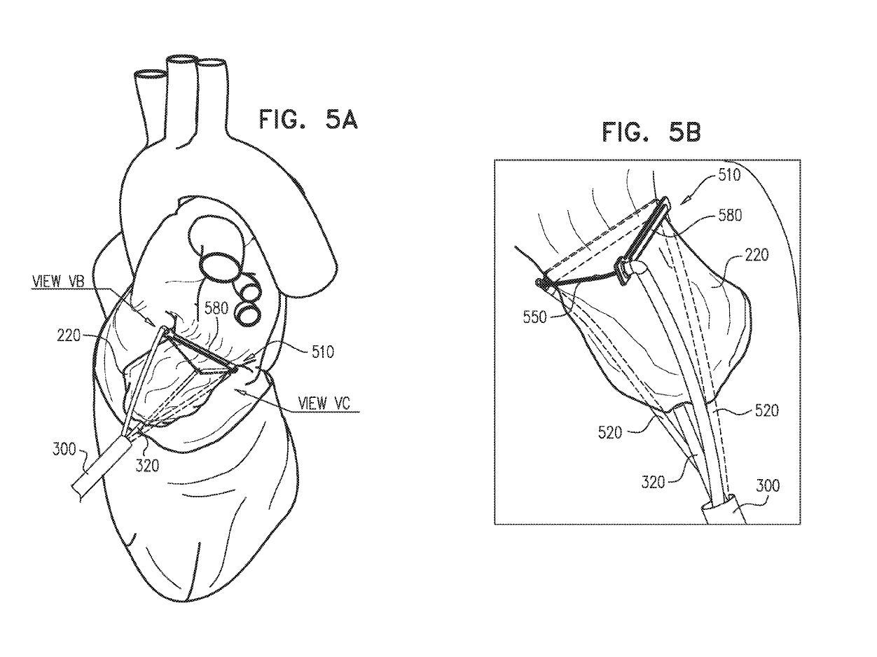

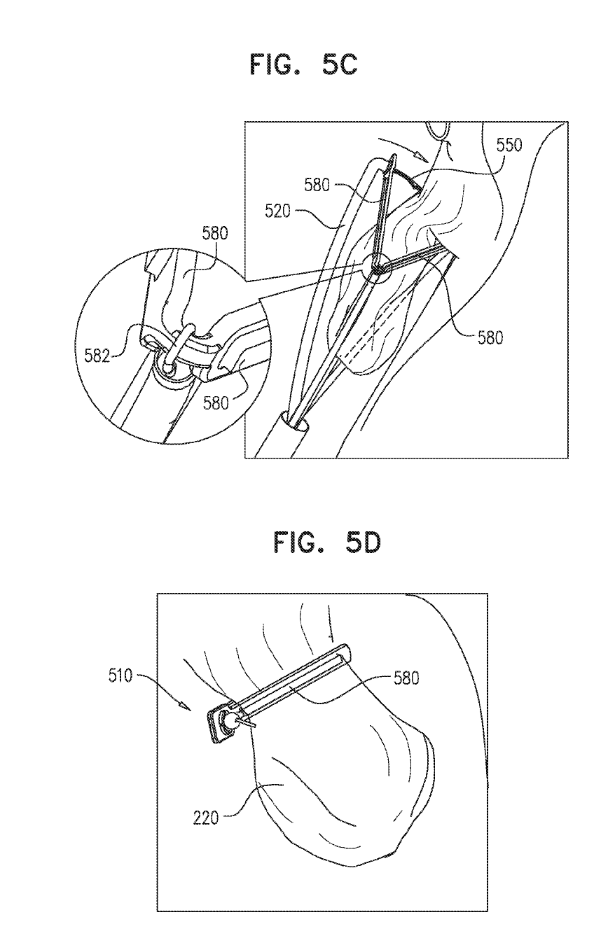

Reference is now made to FIGS. 5A-D, which are schematic illustrations of several views of apparatus 510 for closure of left atrial appendage 220.

Apparatus 510 typically comprises stabilizing tool 320 which is advanced through delivery tool 300 into the pericardial region of the subject following accessing of the pericardium by apparatus and methods described herein with reference to FIGS. 1A-E and/or FIG. 2 (e.g., accessing the pericardial region by use of apparatus 20 or functional equivalents thereof as described). Stabilizing tool 320 is shaped and arranged to stabilize tissue of left atrial appendage 220 typically by grasping the tissue or applying suction thereto.

Apparatus 510 comprises a clip element 580 which is deployed around tissue of LAA 220, e.g., around a base portion of LAA 220. Clip element 580 is shaped to define two longitudinal arms connected by a hinge 582 to form a triangular clip structure. A suture 550 is disposed in a disposition that couples free ends of clip element 580. Clip element 580 is deployed through deployment rods 520 which are advanced into the pericardial region through delivery tool 300. Pulling suture 550 typically closes clip element 580 over the LAA.

Once clip element 580 is securely placed around a portion of LAA 220, stabilizing tool 320 and rods 520 are retracted. Clip element 580 is deployed around a portion of LAA 220 by aligning the clip element not parallel, (e.g., at an angle that is greater than 45 degrees e.g., perpendicularly) to an axis of protrusion of left atrial appendage 220 (FIG. 5D).

Reference is still made to FIGS. 5A-D. For some applications, apparatus 510 comprises a pin or a rivet instead of suture 550.

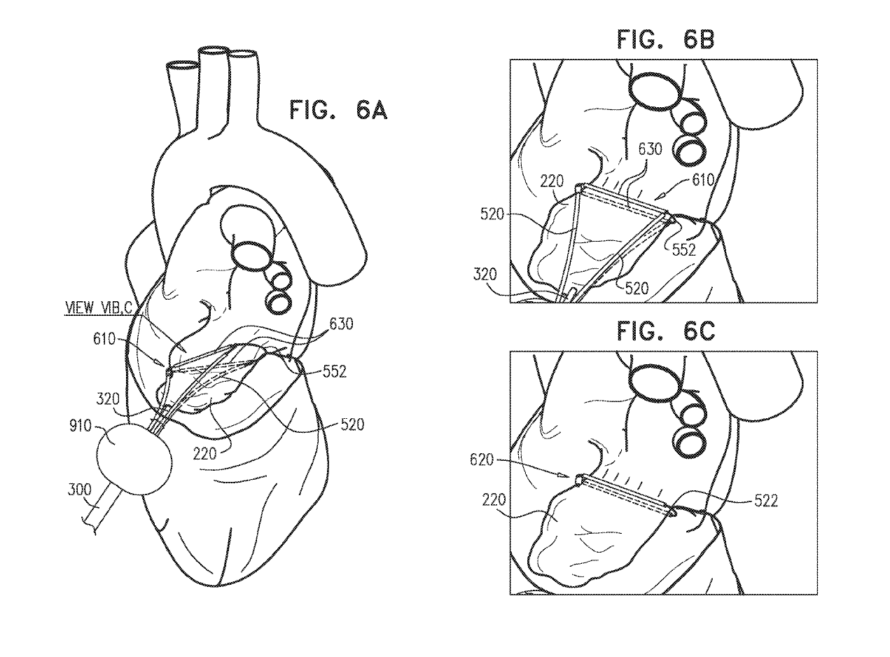

Reference is now made to FIGS. 6A-C, which are schematic illustrations of LAA closure apparatus 610, as provided by some applications of the present invention. Apparatus 610 typically comprises stabilizing tool 320 which is advanced through delivery tool 300 into the pericardial region of the subject following accessing of the pericardial region by apparatus shown in FIG. 1A-E and/or apparatus 200 described in FIG. 2. Stabilizing tool 320 is shaped and arranged to stabilize tissue of left atrial appendage 220, during deployment of apparatus 610, typically by grasping the tissue or applying suction thereto.

For some applications, apparatus 610 comprises first and second longitudinal rod elements 630 which are deployed on opposing surfaces of tissue of LAA 220, e.g., at opposing sides of the base portion of LAA 220. Each rod element 630 is shaped to define a longitudinal arm configured to press on tissue of the LAA to facilitate closure thereof. For some applications, first and second rod elements 630 are rigid. Rod elements 630 are coupled by a coupling suture 552 as shown in FIGS. 6A-C. Pulling coupling suture 552 (from the proximal end (not shown) of delivery tool 300) moves rod elements 630 toward each other, to squeeze the rods against the LAA.

Rod elements 630 are typically positioned via a deployment device, e.g., deployment rods 520, which are advanced into the pericardial region through delivery tool 300. Typically at least one, e.g., two deployment rods 520 (or as shown in FIGS. 6A-B, e.g., three deployment rods 520) is used to deploy apparatus 610.

For some applications, rod elements 630 are each shaped to define flat or rounded tissue-contact surfaces, in order to achieve good contact with tissue of the LAA and to reduce the possibility of injuring tissue of the LAA, while pressing against it with rod elements 630. Once rod elements 630 are securely placed around a portion of LAA 220, stabilizing tool 320 and deployment rods 520 are retracted (FIG. 6C). Typically, rod elements 630 are configured for alignment that is not parallel to an axis of protrusion of left atrial appendage 220, as shown in FIG. 6C. Rod elements 630 are typically deployed generally perpendicularly to an axis of protrusion of the left atrial appendage.

It is noted that for some applications, LAA closure apparatus 610 comprises more than two rod elements 630 (e.g., three rod elements 630, or four rod elements 630) and the rod elements are not necessarily deployed on opposing sides of the LAA. For example, for applications in which there are three rod elements 630, rod elements 630 are positioned around the LAA to form a triangular structure to press and seal the LAA.

For some applications an inflatable element 910 is delivered by, or otherwise coupled to, delivery tool 300 and is disposed within the pericardial region. Inflatable element 910 is typically inflated within the pericardial region in order to facilitate deployment of apparatus 610 by distancing the pericardium from the LAA and enhancing visualization of the LAA. It is noted that inflatable element 910 may be used in combination with any other LAA closure apparatus described herein.

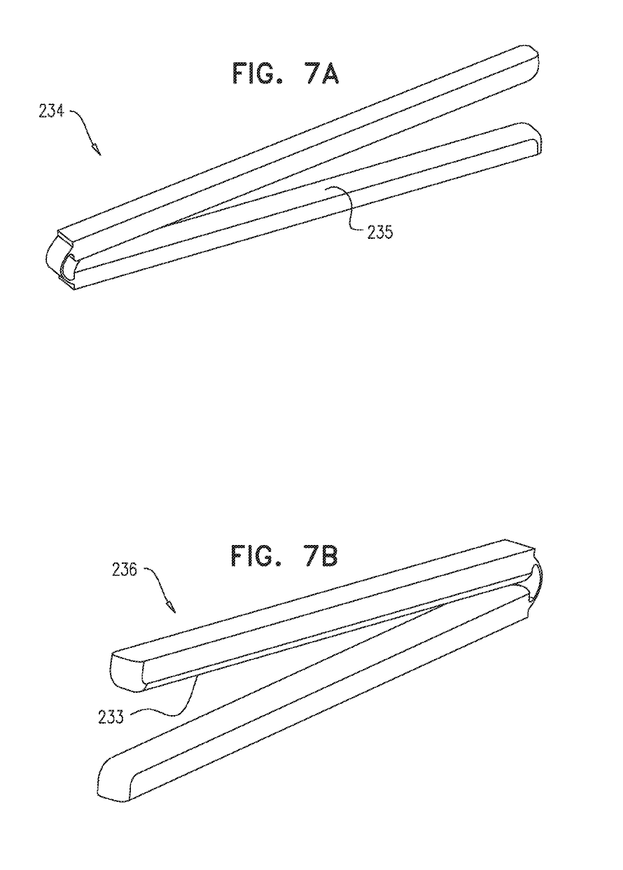

Reference is now made to FIGS. 7A-B, which are schematic illustrations of clip elements 234 and 236 configured for closing the left atrial appendage (LAA), in accordance with some applications of the present invention. Similarly to clip element 580, clip elements 234 and 236 are also configured for alignment that is not parallel to an axis of protrusion of left atrial appendage 220, as described. The clips are typically deployed generally perpendicularly to an axis of protrusion of the left atrial appendage.

It is hypothesized that non-parallel alignment of the clip element achieves enhanced closure of LAA 220 at a base portion of the LAA which is adjacent to the left atrium.

As shown, clip elements 234 and 236 are typically shaped to define tissue-contact squeezing surfaces. For some applications, clip element 234 is shaped to define a flat tissue-contact edge 235, and clip element 236 is shaped to define a rounded tissue-contact edge 233, in order to achieve good contact with tissue of the LAA while pressing against it with the clip elements.

For some applications (not shown), the clip element comprises (a) two rigid longitudinal elements and (b) a cable-tie element having a ratchet mechanism. A “cable-tie element,” in the context of the present application and in the claims, is an element that comprises generally the same structural band and ratchet elements as a standard cable-tie (known in the art of securing devices). Tightening of the clip element around the LAA is typically achieved by tightening the ratchet by moving at least one of the rigid longitudinal elements toward the other rigid longitudinal element. Fastening of the ratchet mechanism of the cable-tie element secures the cable-tie element around the LAA. The cable-tie element closes the LAA typically at the base portion thereof. Typically, a straight, and not curved, surface of the cable-tie element is pressed against a surface of the LAA, thus creating a more secure closure of the LAA. Subsequently to securing the cable-tie element around the LAA, the longitudinal elements are removed from the body of the subject, and the cable-tie element remains secured around the LAA.

The clip element comprising the two rigid longitudinal elements and the cable-tie element having the ratchet mechanism, are described U.S. Provisional Application No. 62/021,327 to Gross et al., entitled “Left atrial appendage closure,” which is incorporated herein by reference.

Reference is made to FIGS. 3A-7B. For some applications, LAA closure apparatus described herein are used in combination with ultrasonic or radiofrequency (RF) scissors used to remove or seal the LAA. It is noted that, in accordance with some applications of the present invention, LAA removal or closure may be performed by ultrasonic or radiofrequency (RF) scissors without use of apparatus described herein.

Reference is still made to FIGS. 3A-7B. Typically, apparatus for closure of the LAA described herein (i.e., apparatus 3100, 410, 510, 610, 234 and 236) are both delivered to the LAA and deployed around the LAA under intracorporeal imaging guidance.

Reference is still made to FIGS. 3A-7B. As described herein, stabilizing tool 320 is configured to hold tissue of the LAA by applying suction or mechanically grasping the tissue. For some applications, tool 320 is additionally configured to hold tissue of the LAA in order to manipulate, e.g., move the tissue, to position the LAA within the closure apparatus. For example, tool 320 may mechanically manipulate tissue of the LAA to facilitate positioning the LAA within suture 310, clip element 580 and/or rod elements 630 (such that suture 310, clip element 580 and/or rod elements 630 are positioned around the LAA).

Reference is still made to FIGS. 3A-7B. Additionally or alternatively to use of stabilizing tool 320, the scope of the present invention includes grasping the LAA by using a magnetic element disposed in an internal cavity of the LAA. Typically an additional magnet, external to the LAA, is used to magnetically grasp the LAA. For some applications, the magnet disposed in the LAA is coupled to a retrieval element, which facilitates removal of the magnet from within the LAA.

Apparatus and techniques described in the following patent applications, each of which is incorporated by reference in the present application, may be combined with apparatus and techniques presented herein:

U.S. patent application Ser. No. 14/704,857 to Gross; and

U.S. patent application Ser. No. 14/324,457 to Gross.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-18. (canceled)

19. Apparatus for closure of a left atrial appendage of a heart, comprising:
a stabilizing tool advanceable into a pericardial region, and shaped and arranged to stabilize tissue of the left atrial appendage;
a closure-suture tube configured to enter the pericardial region and to be advanced toward an outer surface of the left atrial appendage;
a closure suture advanceable out of the closure-suture tube and shaped to define a closure loop configured to close the left atrial appendage by tightening around a base portion of the left atrial appendage;
a control tube configured to enter the pericardial region and to be advanced toward an outer surface of the left atrial appendage; and
at least one control suture advanceable out of the control tube and shaped to define a control loop which is disposed in a disposition looped through the closure suture to facilitate placement of the closure suture at the base portion of the left atrial appendage by the control loop pulling the closure loop.

20. The apparatus according to claim 19, wherein the stabilizing tool is configured to stabilize tissue of the left atrial appendage by applying suction to the tissue.

21. The apparatus according to claim 19, wherein the stabilizing tool comprises a mechanical grasping element and is configured to stabilize tissue of the left atrial appendage by grasping the tissue.

22. The apparatus according to claim 19, further comprising a delivery tool configured in shape and size to enter a pericardial region of the heart and to be advanced toward an outer surface of the left atrial appendage, wherein the apparatus is configured to be advanced into the pericardial region through the delivery tool.

23. A method for closing a left atrial appendage of a heart, comprising:
percutaneously advancing a stabilizing tool into a pericardial region of the heart;
stabilizing tissue of the left atrial appendage with the stabilizing tool;
deploying a closure suture loop around the left atrial appendage, the closure suture loop having a control suture loop looped therethrough;
positioning the closure suture loop around a base portion of the left atrial appendage by pulling the closure suture loop using the at least one control suture loop; and
closing the left atrial appendage by tightening the closure suture around the base portion of the left atrial appendage.

24. The method according to claim 23, wherein advancing the stabilizing tool into the pericardial region comprises advancing the stabilizing tool under the guidance of an intracorporeal imaging device.

25. The method according to claim 23, wherein stabilizing tissue of the left atrial appendage with the stabilizing tool comprises stabilizing the tissue by applying suction to the tissue through the stabilizing tool.

26. The method according to claim 23, wherein stabilizing tissue of the left atrial appendage with the stabilizing tool comprises stabilizing the tissue by grasping the tissue with the stabilizing tool.

27-44. (canceled)
